(19) United States
(12) Patent Application Publication
(10) Pub. No.: US 2019/0393699 A1
(43) Pub. Date: Dec. 26, 2019

(54) Smart Utility Hub

(71) Applicants: Ganesh Shastri, Needham, MA (US); Soundarya Ganesh, Needham, MA (US)

(72) Inventors: Ganesh Shastri, Needham, MA (US); Soundarya Ganesh, Needham, MA (US)

(21) Appl. No.: 16/019,221

(22) Filed: Jun. 26, 2018

(51) Int. Cl. H02J 3/14; H01H 9/02; G01R 22/06; G01N 33/00

(52) U.S. Cl. CPC H02J 3/14; G01N 33/004; G01R 22/068; H01H 9/0271

(57) ABSTRACT

A utility hub is provided. The utility hub is able to control utility outlet flow from an inlet through an outlet based on programming and/or sensed inputs to optimize utility usage. The utility hub uses a computerized controller having a processor and a memory to control utility outlet flow from an outlet.

### BACKGROUND OF THE INVENTION

### Field of the Invention

The present invention relates generally to utility management systems. More particularly, the present invention relates to a computerized and programmable utility management device, such as a utility hub.

### Description of Related Art

Excessive utility usage, such as electrical usage, is a common source of waste, both financially and of the Earth's finite resources. Often, users are not even aware of the extent of the utility waste that they cause. Specifically, electrical draws occur when appliances are not in use, lights are kept on, heat and water runs excessively, and so on. While these wastes may be small when viewed from a single household scale, when viewed from a city, state, or nationwide viewpoint, the totaled amount of waste becomes enormous.

Therefore, there is an opportunity to save waste by being more efficient with the usage and operation of utilities.

### SUMMARY OF THE INVENTION

The subject matter of this application may involve, in some cases, interrelated products, alternative solutions to a particular problem, and/or a plurality of different uses of a single system or article.

In one aspect, a smart utility hub is provided. The utility hub has a computerized controller made up of a processor and a memory. A utility (such as electrical) inlet, and outlet may be connected to a base such as a body or housing of the utility hub and in communication with each other. The computerized controller is operable to track a utility usage passing between the inlet and outlet. The computerized controller is further operable to control a utility usage from the utility outlet upon detection of the utility usage rising above a programmed predetermined expected amount. This expected or desired amount may be programmed into the memory, or control may be influenced by a sensor in communication with the computerized controller.

Often, the expected utility outlet flow amount may be zero, though that is not necessarily required. For example, if a sensor detects that it is night time, and that light levels are above a certain programmed level, and if the utility outlet flow is greater than zero, the computerized controller may shut off utility outlet flow to reduce waste. Further examples are discussed in detail below.

### BRIEF DESCRIPTION OF THE DRAWINGS

FIG. 1 provides a view of one embodiment of the present invention.

FIG. 2 provides a view of another embodiment of the present invention.

FIG. 3 provides a perspective view of still another embodiment of the present invention.

FIG. 4 provides a schematic view of an embodiment of the present invention.

### DETAILED DESCRIPTION

The detailed description set forth below in connection with the appended drawings is intended as a description of presently preferred embodiments of the invention and does not represent the only forms in which the present invention may be constructed and/or utilized. The description sets forth the functions and the sequence of steps for constructing and operating the invention in connection with the illustrated embodiments.

Generally, the present invention concerns a utility hub which allows for monitoring and programmable control of utility output to prevent the waste of energy automatically. The utility hub has a utility inlet, a utility outlet, and a computerized controller operable to control flow through the utility outlet (such as shutting flow on and off) depending on conditions. In electrical applications, the utility hub is operable to track and control excess electrical output, such as “vampire draw,” shutting off appliances when they are not being used as detected by sensors, and when input by a remote networked connection input.

It should be understood that when discussed herein, stopping or slowing flow is presumed to be done when there is a flow through a utility outlet above an expected amount. Most of the noted programmings have an expected utility outlet flow of zero to trigger control, because that is the expected electrical outlet when not in use. However, not all conditions will require this zero expected flow.

The utility hub may be a programmable unit which detects and controls flow of a utility such as electricity, natural gas, propane, heating oil, water, and the like based on a computer controlled system to prevent a waste of the utility as determined by programming of the computerized controller, as well as inputs recorded by a sensor. Control of the flow of the utility may be in the form of stopping and allowing flow, throttling flow rate, and the like.

In a particular embodiment, the utility hub may be an electrical control center. In this embodiment, the utility hub may have an electrical inlet, and one or a plurality of outlets. The outlet(s) may be traditional three prong outlets but are not limited to these. The electrical outlets may be any other electrical outlet configuration as well, such as USB, and the like. In some embodiments, the electrical utility hub may have surge protector between the inlet and outlet.

Various exemplary configurations of the electrical utility hub embodiment are contemplated herein. For example, the outlets may be configured as follows: two traditional outlets, four USB outlets; six traditional outlets, two USB outlets, four traditional outlets, four USB outlets. However, of course the outlet configuration and number may vary without straying from the scope of this invention. In further electrical utility hub embodiments, a rechargeable battery backup may allow for the device to send an alert through an interface on the utility hub, through the outlets, or via a networked connection, and the like. Further, the battery backup may provide a certain amount of time to keep the outlet power flow continuous, allowing a user to make arrangements for the loss of power.

Networked connections may be achieved by a wired connection, Wi-Fi, cellular, Bluetooth®, or other wireless connection. Similarly, the proper transceiver for the wireless network connection may be built into the utility hub and in communication with the computer controller.

The computerized controller of the utility hub is operable to allow each outlet to have its own programming and individualized control. This may be set up as a plurality of different processors, or a single processor. The programming may include input from sensors to influence or control operation to control (such as turn on or off) the utility flow from the outlet. Control to the outlet may be achieved by actuation of a physical switch/valve, or by a virtual controller which can turn on or off the outlet or otherwise control the outlet. Detection of the utility flow through the outlet may be achieved by a meter, or any measurement device/system as is known in the art.

In some further embodiments, a user may be able to access a user interface which may allow for a control of utility hub operations and programming, as well as a tracking interface. The user interface may be accessed through a separate computer through a networked connection with the utility hub, or may be physically on the utility hub. Networked access may be, for example, through a website or mobile app. For example, if the utility hub is an electrical power control center, and a television, computer, and lamp are connected to three outlets, the user interface may allow designation of each outlet according to the appliance plugged into it, and the computerized controller, based on designation, may instruct a particular programming to control operation in a manner optimized for the selected device.

This user interface may be part of the utility hub base (such as a touch screen), or may be a remote user interface such as a computerized login. The user interface allows access and manipulation of the utility hub computerized controller based on the networked connection between user interface and utility hub. The user interface can be used to receive gesture inputs by a user to, among other user interface operations, adjust a programming, and in turn operation, of the utility hub. For example, the user interface may receive inputs to identify and program each of the plurality of outlets. These outlets can be named/tagged. The utility hub has the ability to store an associated “profile” i.e. a programming relating to the specific control and specific sensor inputs to control the outlet. The selection of a profile may correspond to a pre-programmed algorithm optimized for the device corresponding to the profile, which may be loaded to the memory of the utility hub. Non limiting examples of utility outlet profiles may include, but are not limited to: lamps with light measurements, lamps with time restrictions, cell phone chargers, cell phone chargers with Bluetooth® and an app installed to measure battery level; demand-response outlets configured to be turned off at peak usage times; and the like. Such profiles can be updated, modified, added, and the like, based on user feedback and modification.

Pre-set programmings may be available when a user associates a profile to the particular outlet—such as a lamp, DVR, cell phone charger, and the like. Put another way, the user interface allows a user to assign a preconfigured and automated algorithm (programming) to control power utilization through each outlet based on the general characteristics of the device. In many embodiments, the programming may be adjusted (updated and/or changed) remotely through a networked connection to transfer the adjusted programming through the network for saving in the memory. The may allow for adjustment of the operation of the utility hub based on long-term usage patterns. Further, the user interface may be connectable to a voice controlled system and/or a smart home system, such as Amazon® Alexa, Google® Home, Apple® devices, and the like.

Further detailing the control and updating of the programming corresponding to each outlet, in some embodiments, the programming may be in the form of a smart algorithm, which may learn and improve over time through the aggregation of data from the utility hub. This aggregated data may be transferred through the networked interface to a server, which may apply machine learning algorithms to the aggregated data from the utility hub, and optionally aggregated data from other utility hubs. The algorithm used may be optimized and sent for storage in the memory of the utility hub computerized controller through the networked connection. As such, the utility hub may be automatically updated to continuously improve itself.

In a further embodiment of the networked connection, the user interface may be capable of receiving an input to turn each outlet on and/or off manually and remotely. As with other inputs, the user interface receives input and, via networked connection causes instructions to be sent to the computerized controller of the utility hub. In a particular embodiment of remote control, a parental control system may be employed. The parental control embodiment may allow a parent to control the power outlet used by a child, such as by controlling a TV or other electronic device, providing a flashing to warn of impending power shut down, and the like. In still a further embodiment of the user interface, the computerized controller may calculate and be configured to display a power usage information for each outlet over time, with the memory of the computerized controller, or a networked server memory, capable of storing the usage data for later access.

The networked user interface may have additional benefits to engage and retain users. For example, a user, through the networked user interface, may be able to sign up for and receive rebates based on being part of a group of users. For example, a user group may have a goal to reduce power usage at peak times, as coordinated and required by the utility provider. Other engagement options may include people joining a community of like-minded users who seek to optimize energy usage, use rebates for social causes, cash rewards, and the like.

In a particular embodiment of the user interface engagement, the cloud and blockchain networks may be used to ensure the privacy and security of transactions for the earned rebates. Savings and rewards may be earned, such as by reduction of power usage or other goals. Transactions of these savings and rewards may be maintained in cryptocurrencies or as secure transactions. Security may be ensured using blockchains. Power savings, such as those at peak times, can be tokenized so that the utility can identify the total amount of power saved and then reward the users appropriately. The tokens allow the exchange of the savings amounts to other organizations to accept. This, combined with the group engagement discussed above, allows users to “donate” or “encash” their tokens.

The utility hub may have one or a plurality of sensors in communication with the processor. The sensor or sensors are able to detect a condition in the surroundings of the utility hub, and provide an output that, when received by the processor, allows the processor to adjust a control condition of the utility outlet, either through direct control, or by adjusting or changing a programming stored in the memory. Examples of sensors include, but are not limited to, a sound sensor, a light sensor, a motion sensor, a temperature sensor, a carbon monoxide sensor, a clock, and the like.

In an embodiment of the utility hub having a sound sensor, the sensor may be configured to detect sound, or lack thereof, near the utility hub. In turn, the computerized controller processor and memory may be operable to control utility outlet flow based on an amount of detected ambient noise. For example, if silence or low noise is detected, utility outlet flow may be stopped to shut off usage when it appears that nobody is present. Similarly, if a large amount of noise is detected, utility outlet flow may be activated based on a determination that there are people present in the area around the utility hub. In one embodiment, the noise sensor may respond to voice commands. In a particular electrical utility hub embodiment, noise level may be used to automatically shut off electrical outlets drawing power when there is only a minimal amount of ambient noise.

In an embodiment of the utility hub having a light sensor, the sensor may be configured to detect light, or lack thereof, near the utility hub. In turn, the computerized controller processor and memory may be operable to control utility outlet flow based on an amount of detected ambient light. For example, if light levels above a predetermined level are detected, utility outlet flow may be stopped to shut off usage of a lamp when it appears that the lamp is not needed. In some embodiments, utility outlet flow may be controlled based on a programming corresponding to a device connected to the utility outlet. For example, if the detected light is below a predetermined level, the flow to a lamp may be activated. Or, if the detected light is below a predetermined level, electrical flow to an entertainment device may be stopped.

In an embodiment of the utility hub having a motion sensor, the sensor may be configured to detect motion, or lack thereof, near the utility hub. In turn, the computerized controller processor and memory may be operable to control utility outlet flow based on an amount of detected ambient motion. For example, if no or little motion is detected, utility outlet flow may be stopped to shut off usage when it appears that nobody is present. Similarly, if an amount of motion greater than a predetermined amount is detected, utility outlet flow may be activated based on a determination that there are people present in the area around the utility hub. In a particular electrical utility hub embodiment, motion level may be used to automatically shut off electrical outlets drawing power when there is motion below a predetermined level.

In an embodiment of the utility hub having a temperature sensor, the sensor may be configured to detect temperature rising above or below a predetermined temperature near the utility hub. In turn, the computerized controller processor and memory may be operable to control utility outlet flow based on an amount of detected ambient temperature. For example, if temperature rises above a certain temperature, an outlet programmed to control a space heater may be shut off. Similarly, if temperature drops below a certain temperature, an outlet programmed to control an air conditioner or fan may be shut off. Further, if temperature drops below a predetermined amount, it may indicate that a user has reduced the heat and is not occupying the space, and in turn the power flow through an outlet may be stopped. In a further embodiment, the utility hub may be in networked communication (as discussed below) with a network accessible thermostat of a house. In such an embodiment, the computerized controller may be able to have programming adjusted to change the predetermined temperature to match a pre-set temperature of the thermostat when set to an “away” mode.

In an embodiment of the utility hub having a carbon monoxide detector, the sensor may be configured to detect carbon monoxide near the utility hub. In turn, the computerized controller processor and memory may be operable to, for example, cause lights to flash on/off in a specific room or the whole house. As can be understood, other types of alerts or alarms may be available to indicate the detection of carbon monoxide danger.

In an embodiment of the utility hub having a timer sensor, the sensor may be configured to turn on or off certain utility outlets at certain times or after a certain amount of time active. The timer and computerized controller may be independently programmable for timing operation. Also, the timer sensor may be able to turn off a utility outlet after a certain time of usage.

As noted, the utility hub may have any number of various sensors, which may work together or independently, to provide inputs to the computerized controller to impact control of the utility outlet. For example, for a utility outlet programmed for connection to a lamp, the utility outlet may be shut off when ambient light readings are above a certain level and/or when there is no motion detected for a period of time, and/or switching on when ambient light readings are below a certain level.

It is to be understood that while various embodiments are discussed for different components and operations of the utility hub described herein, these different embodiments may be combined in any manner. These varied combinations are all contemplated by the inventor as different aspects of the same invention. Therefore, discussion of different embodiments does not indicate that the parts and configurations are exclusive to the described embodiment. These parts and configurations may be interchanged between embodiments.

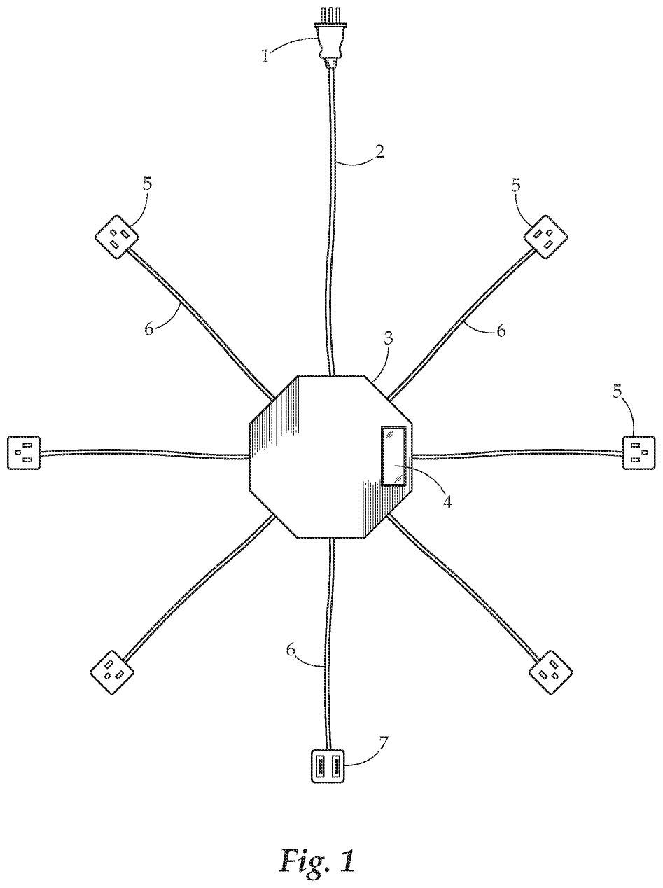

Turning now to FIG. 1, an embodiment of the utility hub, being an electrical controller, is shown. The utility hub has a power inlet 1 which connects to the base 3 via a cord 2. The computerized controller (not shown) is contained within the base 3. A sensor 4, shown here as a light sensor, is in communication with the computerized controller and can provide an input to the controller to inform operation of the utility hub (as discussed above). A plurality of utility outlets, shown here as power outlets 5, extend from the base 3. In this embodiment, each outlet 5 is connected to the base electrically by a cord 6. In some embodiments, the cord 6 may be retractable to allow the outlet 5 to be drawn into and away from the base 3 or to be remotely located from base 3. In a particular embodiment, the cord 6 may be removable, allowing for adjustment of the type and number of outlets. Further, a USB connector 7 may also be connected to the base 3 which can allow electrical flow through a USB (or other related) outlet connection. Each of the power outlets 5 and USB connector 7 is controlled and controllable by the computerized controller.

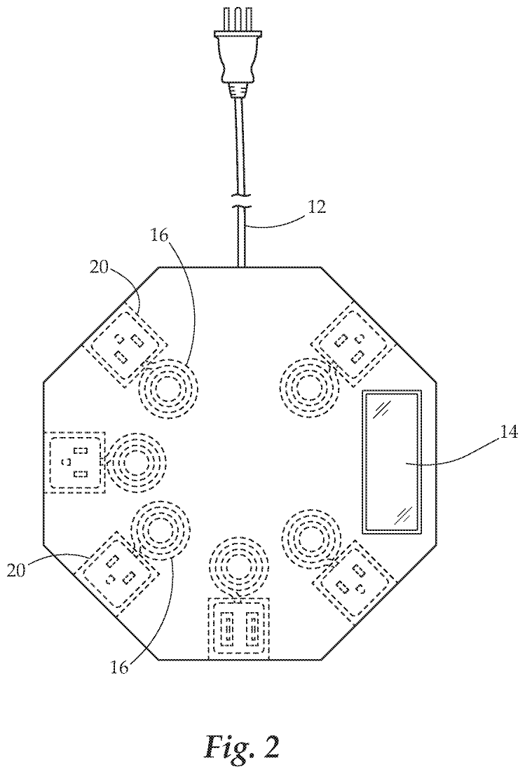

FIG. 2 shows another embodiment of utility hub configured as an electrical controller. As in FIG. 1, there is a power inlet 12 which connects to the base of the utility hub. The computerized controller (not shown) is contained within the base. A sensor 14, shown here as a light sensor, is in communication with the computerized controller and can provide an input to the controller to inform operation of the utility hub (as discussed above). Power outlets 20 are housed within the base, and can be drawn away from the base via the spooled cables 16 which both connect the power outlets 20 to the base, and provide electrical communication thereto.

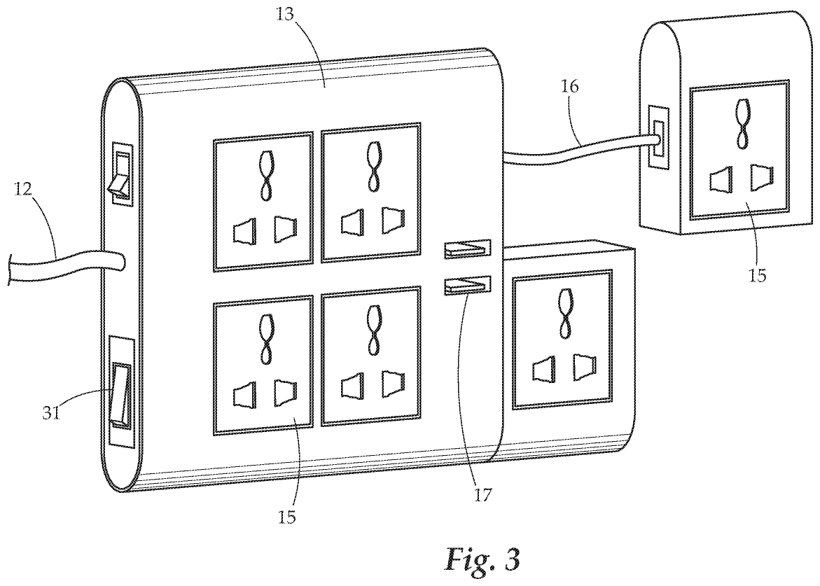

FIG. 3 shows yet another embodiment of the utility hub. In this view, the utility hub has a power inlet 12 enters the base 13. A computerized controller (not shown) is housed within the base 13 and controls operation of the power outlets 15, 17. In this embodiment, some power outlets 15, 17 are mounted to the base 13, while others are retractable and extendable from the base and connected by cable 16. As discussed above, a sensor or sensors may be connected to the base and in communication with the computerized controller. Based on inputs from the various sensor(s), and depending on configuration of the computerized controller, the power outlets 15, 17 may be shut down to optimize and minimize power usage.

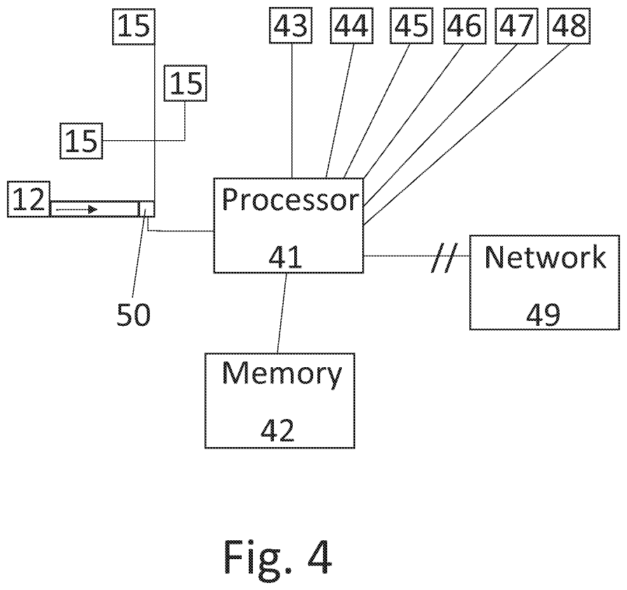

FIG. 4 provides a schematic view of an embodiment of the present invention having various optional sensors and features. The utility hub has a processor 41 and a memory 42 programmed to cause operation of the processor 41. A power inlet 12 has a controller, such as a switch 50, which can be controlled by processor 41. Electricity (or other utility flow) can pass to outlets 15 when the controller 50 allows it, and is prevented from reaching the outlets when prevented by controller 50. As noted above, the processor 41 may be in communication with a network 49 which can provide inputs and receive outputs from the processor 41, can reprogram or update the processor 41 programming (stored in the memory 42) and so on as discussed above, to optimize and update power usage programming. A plurality of sensors are in communication with the processor. In varying embodiments, none, only one, multiple, or all of the sensors may be connected to the processor. Sensors shown include a light sensor 43, a sound sensor 44, a motion sensor 45, a temperature sensor 46, a carbon monoxide sensor 47, and a timer/clock 48. Each of these can provide an input in the processor 41 which can impact operation of the processor's 41 usage of the controller 50. Also of note is that while in this view, the controller 50 is a single controller which limits operation of multiple outlets 15, each individual outlet 15 may be separately controllable by the processor 41 without straying from the scope of this invention. It should be understood further that the sensors 43-48 may be physically separate from the utility hub, or connected to the utility hub while still communication with the controller 50. Moreover, the utility hub base may not necessarily be physically connected to the outlets. As shown in some embodiments, a wire or cable may connect, directly or indirectly, the outlet to the utility hub and inlet.

While several variations of the present invention have been illustrated by way of example in preferred or particular embodiments, it is apparent that further embodiments could be developed within the spirit and scope of the present invention, or the inventive concept thereof. However, it is to be expressly understood that such modifications and adaptations are within the spirit and scope of the present invention, and are inclusive, but not limited to the following appended claims as set forth.

What is claimed is:

1. A smart utility hub comprising:
a computerized controller comprising a processor, and a memory within a base;
a utility inlet;
a utility outlet; and
wherein the processor of the computerized controller is operable to track a utility usage through the utility outlet, and operable to control a utility flow from the utility outlet based on a programming stored in the memory, the control programming based on detection of the utility flow above an expected programmed amount and an input from a sensor in communication with the processor.

2. The smart utility hub of claim 1 wherein the utility is at least one of electricity, natural gas, propane, heating oil, and water.

3. The smart utility hub of claim 1 wherein the utility is electricity, and wherein the utility outlet is one of an electrical outlet and a USB outlet.

4. The smart utility hub of claim 1 wherein the processor of the computerized controller is operable to control the utility usage by actuation of at least one of a switch or a valve.

5. The smart utility hub of claim 1 further comprising a plurality of sensors in communication with the processor.

6. The smart utility hub of claim 5 wherein the plurality of sensors comprises at least one of: a sound sensor, a light sensor, a motion sensor, a temperature sensor, a carbon monoxide sensor, and a clock.

7. The smart utility hub of claim 1 further comprising a plurality of utility outlets, and wherein the processor is capable of individually controlling each of the plurality of utility outlets based on a programming assigned to each of the plurality of utility outlets.

8. The smart utility hub of claim 7 wherein the programming assigned to each of the plurality of outlets is stored in the memory, and is adjustable.

9. The smart utility hub of claim 8 further comprising a network connection providing communication to the processor, and wherein the programming assigned to each of the plurality of outlets is adjustable based on an input from the networked connection.

10. The smart utility hub of claim 8 wherein the programming assigned to each of the plurality of outlets is adjustable based on a device connected to each of the plurality of outlets.

11. The smart utility hub of claim 9 wherein the processor is operable to control a utility outlet flow through each of the plurality utility outlets based on an input from the networked connection.

12. The smart utility hub of claim 7 wherein each of the utility outlets is connected to a base of the smart utility hub.

13. The smart utility hub of claim 7 wherein the computerized controller is operable to track a utility usage through each of the plurality of utility outlets, and operable to control a utility flow from each of the plurality of utility outlets individually based on the programming stored in the memory, the control programming based on the detection of the utility flow above the expected programmed amount and an input from the sensor in communication with the processor.

14. The smart utility hub of claim 1 wherein the sensor is a light sensor, and wherein the processor is operable to control utility usage through the utility outlet based on a detected ambient light level.

15. The smart utility hub of claim 1 wherein the sensor is a temperature sensor, and wherein the processor is operable to control utility usage through the utility outlet based on a detected temperature.

16. The smart utility hub of claim 1 wherein the sensor is a sound sensor, and wherein the processor is operable to control utility usage through the utility outlet based on a detected sound level.

17. The smart utility hub of claim 1 wherein the sensor is a motion sensor, and wherein the processor is operable to control utility usage through the utility outlet based on a detected motion or lack of motion.

18. The smart utility hub of claim 1 wherein the sensor is a timer, and wherein the processor is operable to control utility usage through the utility outlet based on a passage of time.

19. The smart utility hub of claim 1 wherein the sensor is a carbon monoxide sensor, and wherein the processor is operable to adjust utility usage through the utility outlet based on a detected carbon monoxide level.

20. The smart utility hub of claim 1 wherein the control of the utility usage comprises stopping a utility outlet flow from the utility outlet.
